(19) United States
(12) Patent Application Publication
(10) Pub. No.: US 2025/0099638 A1
(43) Pub. Date: Mar. 27, 2025

(54) ULTRAVIOLET SHOE SANITIZER

(71) Applicant: Megan T. Liu, Dunwoody, GA (US)

(72) Inventor: Megan T. Liu, Dunwoody, GA (US)

(21) Appl. No.: 18/893,719

(22) Filed: Sep. 23, 2024

Related U.S. Application Data
(60) Provisional application No. 63/540,140, filed on Sep. 25, 2023.

(51) Int. Cl. A61L 2/10; A47L 23/02; A61L 2/24

(52) U.S. Cl. CPC A61L 2/10; A47L 23/02; A61L 2/24; A61L 2202/11; A61L 2202/121; A61L 2202/14; A61L 2202/16

(57) ABSTRACT

A shoe sanitizer that uses ultraviolet light sources to reduce or eliminate shoe odor. The shoe sanitizer comprises a cover connected to a central body that comprises one or more ultraviolet light sources. The central body can be extendable to accommodate shoes having different dimensions.

### CROSS-REFERENCE TO RELATED APPLICATIONS

This application is a non-provisional application claiming the benefit of U.S. Provisional Application Ser. No. 63/540,140 filed Sep. 25, 2023 entitled “Ultraviolet shoe sanitizer,” the entirety of which is hereby incorporated by reference.

### TECHNICAL FIELD

The present application relates generally to the field of shoe sanitizers capable of reducing shoe odors.

### BACKGROUND

According to a research study, the 2012 National Foot Health Assessment, conducted for the Institute for Preventative Foot Health, 16 percent of adults age 21 and older, a population of about 36 million people, have experienced foot odor. (https://www.ipfh.org/foot-conditions/foot-conditions-a-z/foot-odor). According to an article published by the American Society for Microbiology, foot odor is caused from microbes on the skin that metabolize compounds in sweat, which produces offensive odors in the form of volatile organic compounds, or VOCs. Some of these skin microbes comprise Corynebacterium, Staphylococcus and Cutibacterium genera. (https://asm.org/Articles/2021/December/Microbial-Origins-of-Body-Odor). When sweat is left behind in shoes, this can result in shoes that also have an odor. (https://www.verywellfit.com/ways-to-prevent-stinky-shoes-smelly-feet-3436314). High levels of harmful microorganisms sustained in enclosed shoes may cause or promote various foot ailments.

Many solutions have been used to alleviate the issue of shoe odor. Such solutions have included washing the shoes and insoles, dowsing the shoes with odor-absorbing powders, spraying shoes with a disinfecting spray, or placing objects inside to try to absorb and mask the odor.

It is well known that ultraviolet (UV) light of certain wavelengths, intensities, and durations can destroy or inhibit growth of surface pathogens. (https://en.wikipedia.org/wiki/Ultraviolet_germicidal_irradiation). One existing solution uses a shoe tree structure having ultraviolet light emitters that can be placed into a shoe. This approach to addressing shoe odor includes disinfecting the shoe with UV light generated from UV light emitting diodes (LEDs) that are mounted over an inside of a hollow shoe tree. With the shoe tree structure, the ultraviolet (UV) light is not directed to both the front and back of the insides of the shoe. The sanitizer also appears large and heavy, and expensive (as of this application's filing date, approximately $100).

Another ultraviolet-based solution, described in U.S. Pat. No. 10,314,928 uses a shoe sole insert that can be an insole, a footbed enclosure, or the like that has UV light sources embedded in the insert; the insert is configured to emit UV radiation in the footwear through a transparent window region formed in the insert. The background of '928 mentions the shoe tree using UV LEDs. The background of the '928 also mentions an enclosure to contain UV light emanating from a bulb inserted inside a shoe without the support of a shoe tree. The background also discusses safety issues so that UV light does not escape the shoe.

The above-described background relating to shoe deodorizing is merely intended to provide a contextual overview and is not intended to be exhaustive. Other contextual information may become apparent in view of the following description.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 illustrates a side view of an example shoe sanitizer, in accordance with various aspects and example embodiments of the subject application.

FIG. 2 illustrates a side view of protruding UV LEDs, in accordance with various aspects and example embodiments of the subject application.

FIG. 3 illustrates a front view of the shoe sanitizer, in accordance with various aspects and example embodiments of the subject application.

FIG. 4 illustrates the coupling of the cover of the shoe sanitizer with the shoe collar, in accordance with various aspects and example embodiments of the subject application.

FIG. 5 illustrates a top view of the shoe sanitizer, in accordance with various aspects and example embodiments of the subject application.

FIG. 6 illustrates an isometric view of the shoe sanitizer, in accordance with various aspects and example embodiments of the subject application.

FIG. 7 is an illustration of an example of a single-member central body of the shoe sanitizer, in accordance with various aspects and example embodiments of the subject application.

FIG. 8 illustrates openings in the central body of the shoe sanitizer through which UV light can pass, in accordance with various aspects and example embodiments of the subject application.

FIG. 9 is an illustration a connector and central body that are extendable, and connected to each other at an angle that can be adjusted, in accordance with various aspects and example embodiments of the subject application.

FIG. 10 illustrates another example in which the central body is connected to the cover of the shoe sanitizer, in accordance with various aspects and example embodiments of the subject application.

FIG. 11 illustrates an example of circuitry of the shoe sanitizer, in accordance with various aspects and example embodiments of the subject application.

FIG. 12 illustrates another example of circuitry of the shoe sanitizer, in accordance with various aspects and example embodiments of the subject application.

### DETAILED DESCRIPTION

The subject disclosure is now described with reference to the drawings, wherein like reference numerals are used to refer to like elements throughout. The following description and the annexed drawings set forth in detail certain illustrative aspects of the subject matter. However, these aspects are indicative of but a few of the various ways in which the principles of the subject matter can be employed. Other aspects, advantages, and novel features of the disclosed subject matter will become apparent from the following detailed description when considered in conjunction with the provided drawings. In the following description, for purposes of explanation, numerous specific details are set forth in order to provide a more thorough understanding of the subject disclosure. It may be evident, however, that the subject disclosure can be practiced without these specific details. In other instances, structures and devices are shown in block diagram form to facilitate describing the subject disclosure. Moreover, components are not necessarily shown to be at scale. Components can be larger or smaller, or longer or shorter than depicted in the drawings.

In accordance with various aspects and example embodiments of the subject application, a shoe sanitizer is provided that can emit ultraviolet (UV) radiation (also referred to as UV light) when a portion of the shoe sanitizer is disposed within the cavity of a shoe where a foot would normally be.

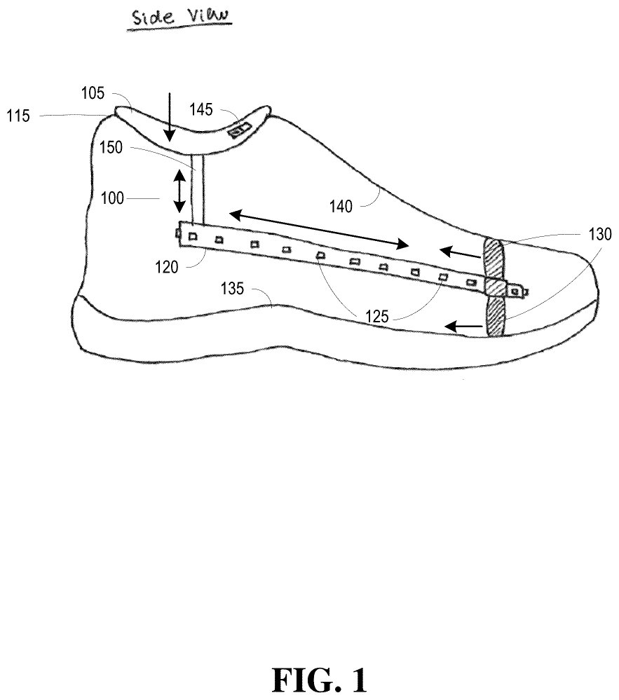

FIG. 1 shows a side view of a shoe sanitizer 100 and a cross sectional view of a shoe with the shoe sanitizer 100 disposed within the inside of the shoe. The shoe sanitizer 100 can comprise (meaning include but not limited to) a cover 105 that fits over the opening of the shoe (i.e., mouth of the shoe) in which a person's foot enters, the opening defined by what is often referred to as the collar 115 and also defined by the top portion of a tongue (if a shoe has a tongue). Those of ordinary skill may sometimes refer to the collar as the top line, or the cuff. The cover 105 of the shoe sanitizer 100 has attached to it a central body 120 of the shoe sanitizer 100. The central body 120 can have one or more UV sources 125 associated with the central body 120.

The central body 120 can be, for example, a rectangular body, or a cylindrical body, and can be made of plastic or some other durable material. It can, for example, be pieces of molded plastic joined together (for example two halves of molded plastic) which result in a single member. It can be large enough to house electronic components inside of it; some of these components will be described below. For example, either the cover 105 or the central body 120 can comprise a battery as its power source. If most of the electronic components will be embedded within the cover 105, then the central body 120 can be thinner. As shown by the two-way arrow next to the central body 120 in FIG. 1, the central body 120 can also be extendable, elaborated further below.

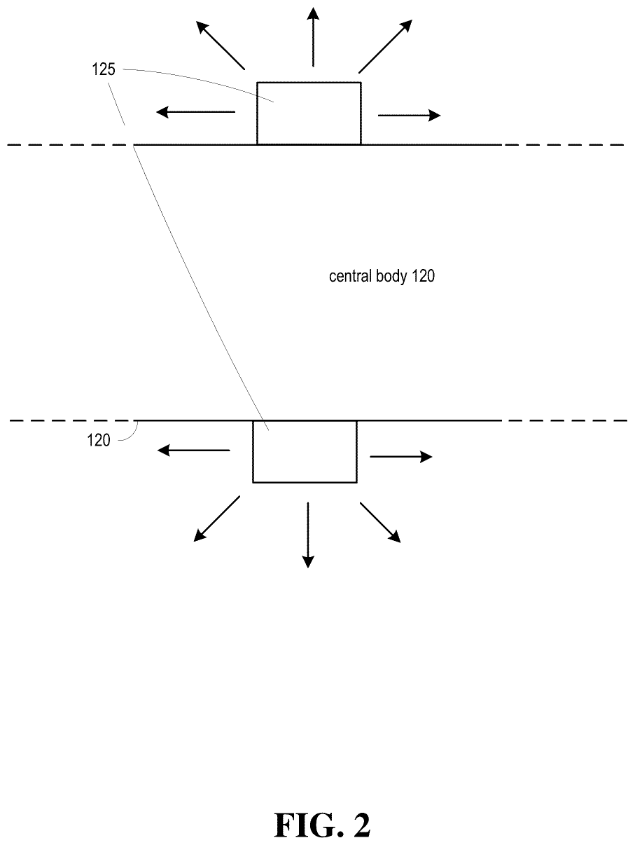

The UV sources 125 can emit electromagnetic radiation within a certain range that is effective to destroy microorganisms in the shoe. The UV sources 125 can be a UV LED, UV bulb, or strip of material capable of emitting UV light. In alternative example embodiments, infrared light that kills microorganism can be used as well. In example embodiments, as shown in FIG. 1, the UV sources 125 can be UV LEDs that are rectangular or squarish in shape. UV LEDs having short-wavelength UV (UV-C) are considered “germicidal UV.” The UV LEDs can be positioned anywhere on the central body 120 such that the reach of the UV light emitted by the UV sources 125 can reach portions of the front, bottom, top, and back of the inside of the shoe. The UV LEDs can thus be located on the sides, top, bottom, back, of the central body 120, and can be located 180 degrees, 90 degrees, or 120 degrees apart from each other. FIG. 1 shows a view in which one can see the UV sources 125 located on a side of the central body 120. In example embodiments, the UV sources 125 are not recessed, such that they protrude outward, as shown in FIG. 2, allowing them to generate UV light emissions in multiple directions. In FIG. 2, the UV LEDs are 180 degrees apart from each other, on opposite sides of the central body 120. In other example embodiments the UV sources 125 can be recessed, wherein each UV source can be disposed such that reflective material (e.g., mirrors, polished metal, reflective coating) around the UV sources 125 can serve to direct emissions in various directions. The number of the UV LEDs can vary, depending on the intensity and range of the spectrum used. For example, with more intensity, fewer UV LEDs may be used.

The shoe sanitizer 100 can also have one or more stabilizers 130 that can impinge on the insole 135 of the shoe. The insole 135 can come with the shoe when purchased or acquired, or can be a separate insert, which can be subsequently acquired or purchased, that is inserted into the shoe for extra cushioning, arch support, etc. In example embodiments, the stabilizers 130 can serve to keep the front portion of the central body 120 from touching or getting too close to the insole 135. In some example embodiments, one or more stabilizers 130 can also be oriented upward such that they impinge on the inside of the shoe's ceiling 140 (which can comprise the tongue of the shoe, laces, or lace hole liners), thus stabilizing the central body 120 such that it is not impinging on the insole 135 or the ceiling 140. In example embodiments, one stabilizer can be used, wherein the base of the stabilizer is broader and resembling the base of a triangle. The base and angle can be curved such that the curved portion corresponding to the angle of the triangle can be in contact with the insole 135. The stabilizers 130 can be made of deformable or flexible material, such that the stabilizers 130 can bend, or curve, as shown by the arrows near the stabilizers 130. Like the cover 105, the material for the stabilizers 130 can comprise, for example, silicon, plastic, rubber, or flexible metal surrounded by silicon, plastic, or rubber. The stabilizers 130 can be made of transparent, or translucent material, such that if they are in the path of any LED's emitted light, then the light can still pass through.

The shoe sanitizer 100 can also comprise a connector 150, which can on one end be connected to the cover 105, and on the other be connected to the central body 120. The connector 150 can serve as a conduit, which can be made of plastic, through which one or more conductors (e.g., wires) carry electrical signals (e.g., power, current) between components associated with the cover 105 and components associated with the central body 120. The shoe sanitizer 100 can also comprise a switch 145, which a user can use to turn the shoe sanitizer 100 on or off, which results in the UV sources 125 emitting UV light, or when turned to the off position, ceasing to emit the UV light. Power to the UV sources can come from an AC-DC power adapter that draws power from an electrical socket, or the power source can be a battery, which can be rechargeable. The switch 145 can be of any kind, for example, a switch that moves from an on to off position, or a button that depresses to an on state, or a button that toggles between on and off. The user might be instructed to keep the UV sources turned on for 1 minute, 2 minutes, 5 minutes, 10 minutes, etc., depending on the type of UV sources 125, in terms of the characteristics and intensity of the emitted UV light. As shown by the two-way arrow next to the connector 150 in FIG. 1, the connector 120 can also be extendable, elaborated further below.

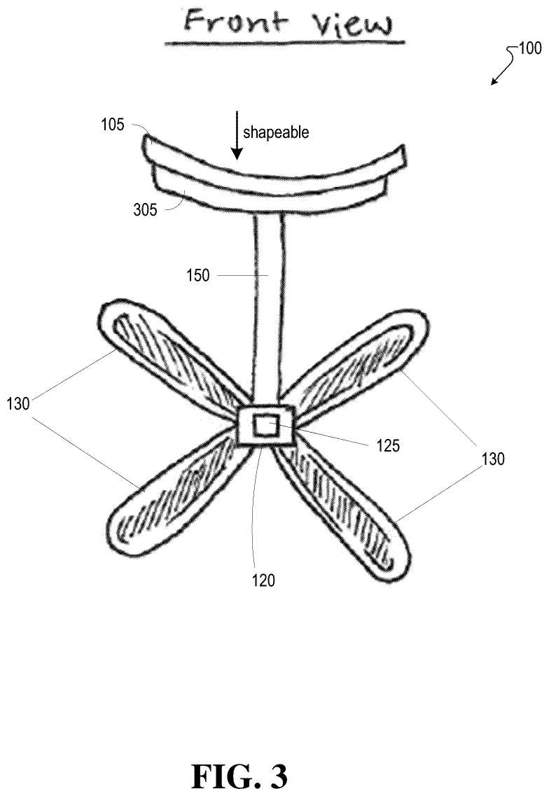

FIG. 3 illustrates a front view of the shoe sanitizer 100 in accordance with example embodiments. The cover 105 may have attached to it a seal 305, which can be, for example, a cylindrical ring. The seal 305 can be made of rubber, foam, or the like. As shown by the arrow, the cover 105 can be shapeable. It can be made of deformable material so that it can, for example, take the shape of the opening of any shoe, deforming to rest on the collar 115. Alternatively, the cover 105 can be made of a flexible material that can be bent to accommodate the opening of any shoe. Material for the cover 105 can comprise, for example, silicon, plastic, rubber, or flexible metal surrounded by silicon, plastic, or rubber. In example embodiments, the cover 105 can also comprise a fabric or cloth skirt (which is opaque) that can fit over the collar 115 of the shoe. Particularly for high-top sneakers and boots, this fit can be more practical. Additionally, the cover 105 can have attached to it, or embedded within it, a power source such as a battery, which in some example embodiments can be flatter. The cover 105 can also have an interface for connecting the hand sanitizer 100 to an AC power adapter.

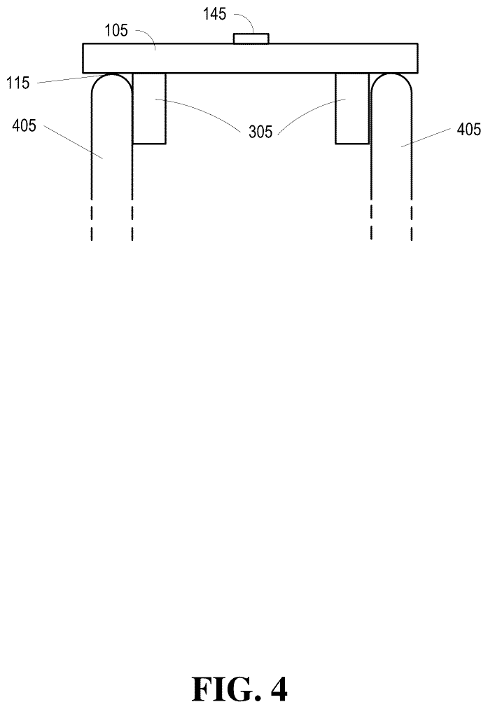

FIG. 4 illustrates another front view with focus on where the cover 105 meets the collar 115. The outer wall of the seal 305 can be in contact with inside of the walls 405 of the shoe. Laces of the shoe can be adjusted to enable a better fit. The fit need not be perfect (e.g., no need to be a tight fit); it can be just enough that the cover 105 does not move around too much or gets out of position. The walls of the cylinder-shaped seal 305 can be made to be long enough such that no direct UV light from the UV sources 125 can exit the shoe through the shoe's mouth. In example embodiments, the front of the central body 120 can also have one or more UV sources 125, which may be conducive if the stabilizers 130 are behind the front UV LED and if the stabilizers 130 are not translucent enough, such that emitted UV light from UV sources 125 located on the central body 120 behind the stabilizers 130 cannot get through. In this scenario, the UV source or sources located in the front of the central body 120 can still emit UV radiation that will reach the front part of the shoe where the toes are.

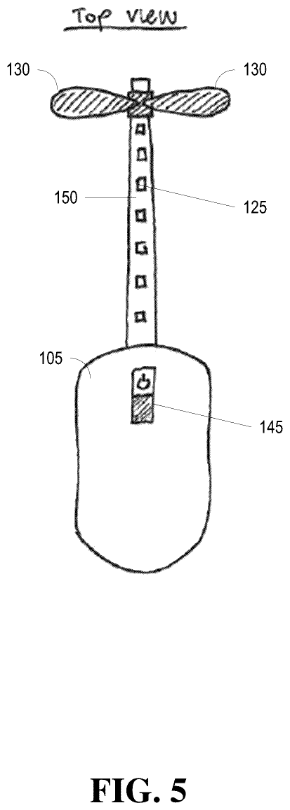

FIG. 5 shows a top view of the shoe sanitizer 100.

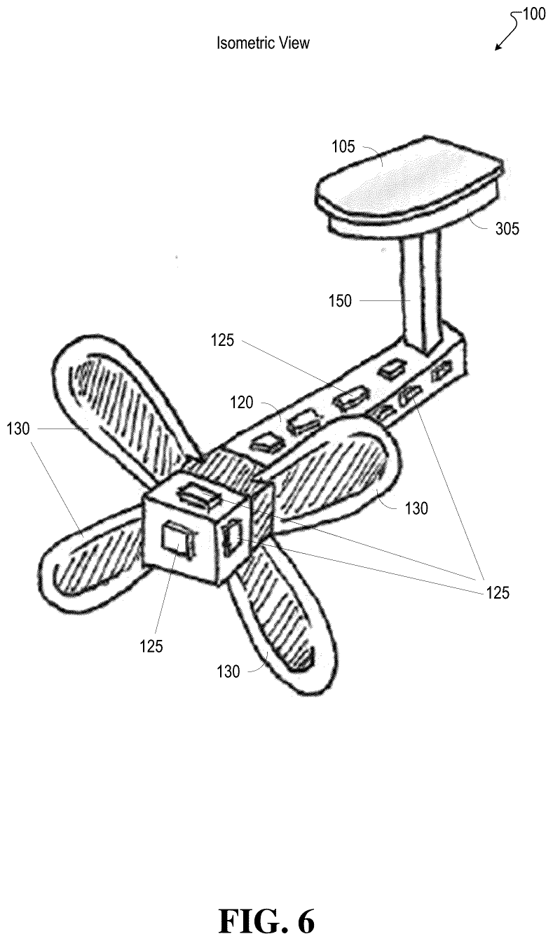

FIG. 6 shows an orthographic projection (isometric) view of the shoe sanitizer 100.

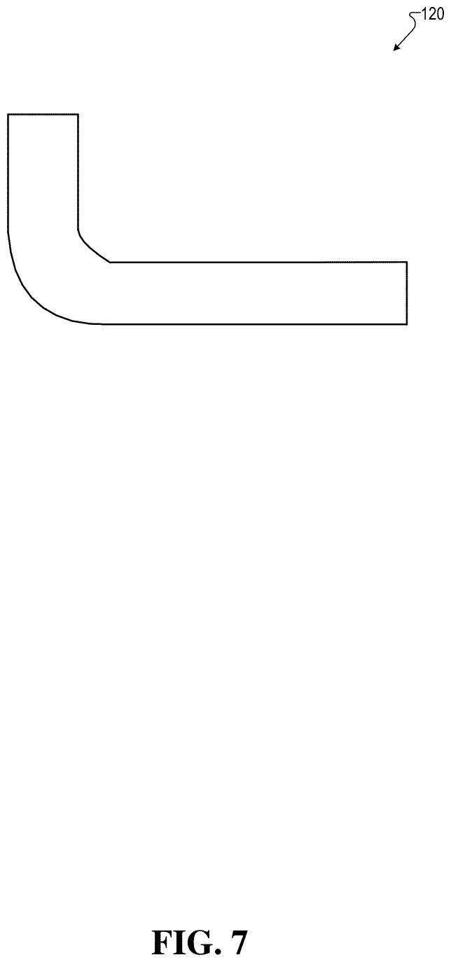

FIG. 7 shows a side view of a central body 120 in which the central body 120 can be one piece. This one-piece member can still result from two symmetrical molded plastic halves joined together. Here, the single member serves as both the central body 120 and the connector 150.

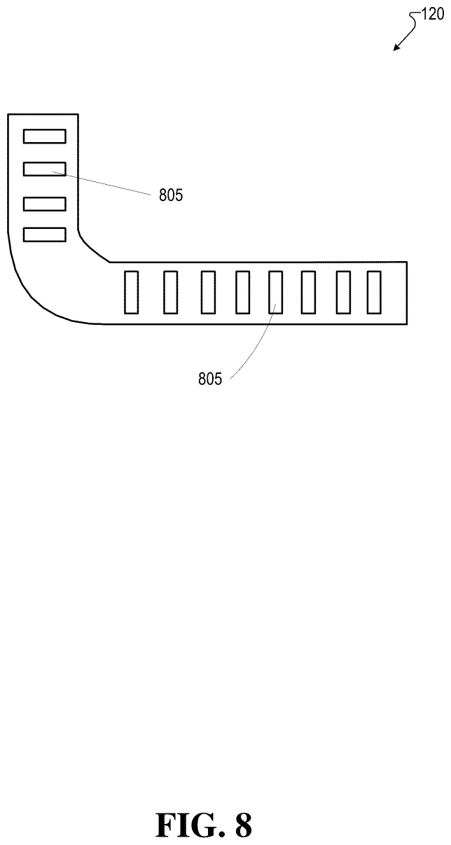

FIG. 8 shows a side view of a central body 120 with openings 805, which can be on the sides, top, bottom, front, back, etc. The openings 805 can be holes, slits, or slots, which can be rectangular, circular, oval, square, or other shapes. The connector 150, or portions of the single-member central body 120 corresponding to the connector 150, can also have openings 805. In example embodiments, the UV light sources can be disposed within the central body 120 or the connector 150, and the UV light emitted from the UV light sources 125 can pass through the openings and reach different parts of the inside of the shoe. Alternatively, the openings 805 can instead be translucent material that can allow UV light to pass (such as a translucent plastic window). The quantity, shape, and size of the openings 805 can also differ in accordance with design preferences, which can be dictated by the intensity and range of the UV light emitted. In example embodiments, the central body 120, connector 150, or portions of the central body 120 or the connector 150, can be made of translucent material (e.g., plastic), wherein UV light sources 125 can pass through the central body 120, the connector 150, or the portions made of translucent material.

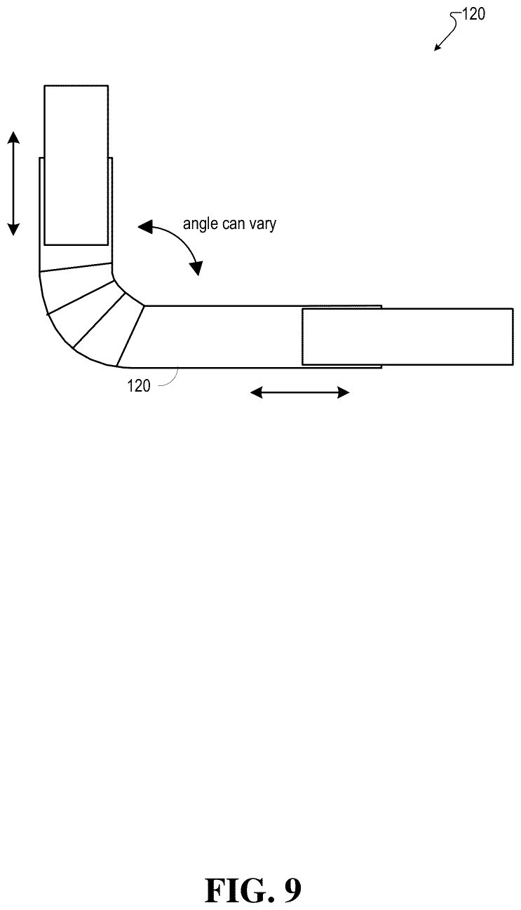

FIG. 9 shows a side view of the central body 120 depicting various aspects in accordance with example embodiments of the present invention. In some example embodiments, the central body 120 can extent and contract, to accommodate shoes of varying size. This can be accomplished, for example, by having two parts to the central body 120. The two parts can be slidably in contact with each other, and slide to lengthen or shorten the central body 120. Friction or notches, or any other technique known to those of ordinary skill, can be used to maintain the length of the central body 120. Particularly for boots and high-top sneakers, a longer connector portion can be desired, which can be achieved by designing several shoe sanitizers 100 each having a connector 150 of a different length, or by using a variable connector 150 as shown in FIG. 9. While not shown in the figures, the connector 105, or in the case of FIG. 7, the portion of the central body 120 that serves as the connector 150, can also have UV sources 125, which can be more effective especially for boots or high-top sneakers. Thus, example embodiments can have a connector 150 that can extend, or a central body 120 that can extend, or both.

Staying on FIG. 9, in example embodiments, the angle at which the connector 150 meets with the central body 120 can also be variable, either by way of manufacture, or, in the case of a single member body as shown in FIG. 7, with an accordion-like flexible junction area.

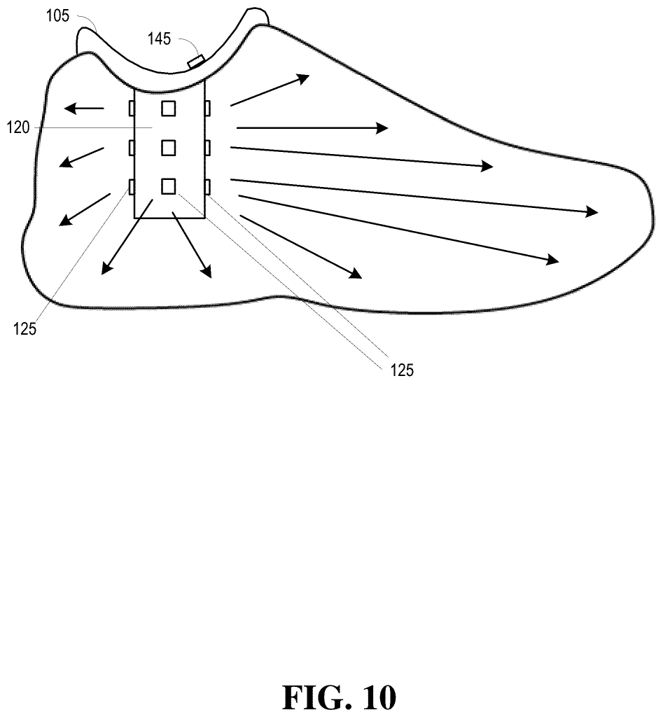

FIG. 10 illustrates example embodiments in which a vertical central body 120 is attached to the cover 105. In these embodiments, the UV sources 125 are selected to have emissions that are intense enough that the UV radiation can reach the front of the shoe, and as such, does not need an extended horizontal member or component. As with FIG. 8 and FIG. 9, the central body can be designed to be extendable, and have openings 805.

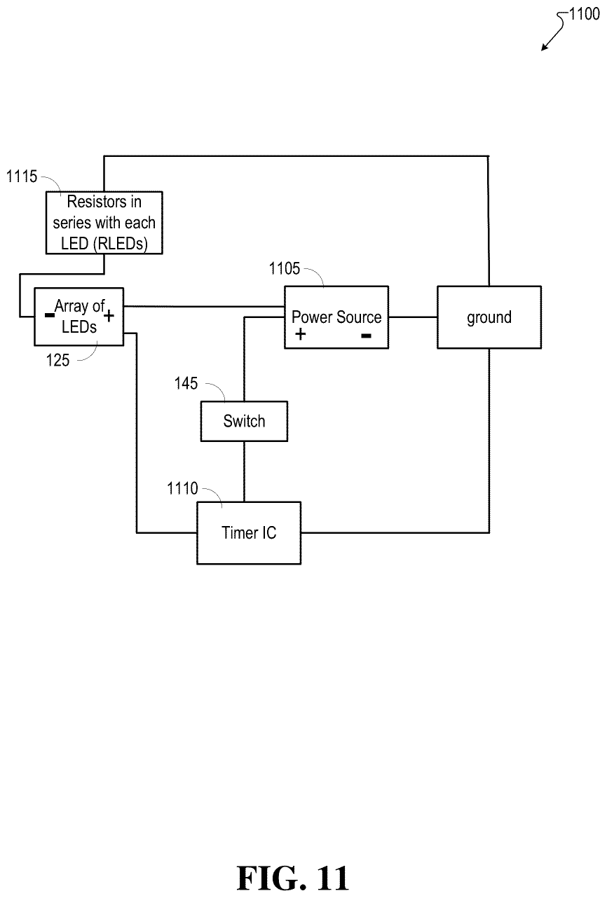

FIG. 11 illustrates an example of a circuit 1100 that can be used to operate the shoe sanitizer 100, in accordance with example embodiments. Unlike as described above in which the user can turn the UV sources 125 on or off manually, the circuit 1100 described in FIG. 11 can use a timer that can control how long the UV sources 125, in this case UV LEDs, stay on. In the example of FIG. 11, when switch 145 is depressed, power flows from a power source 1105 (e.g., a battery) to the UV LEDs. A timer integrated circuit, timer IC 1110, can control how long the UV sources 125 stay on and continue to emit UV light. Current-limiting resistors 1115 can be coupled to the UV LEDs, which can prevent damage to the UV sources 125 by facilitating their operations within their specified current range so they do not get damaged due to excessive current.

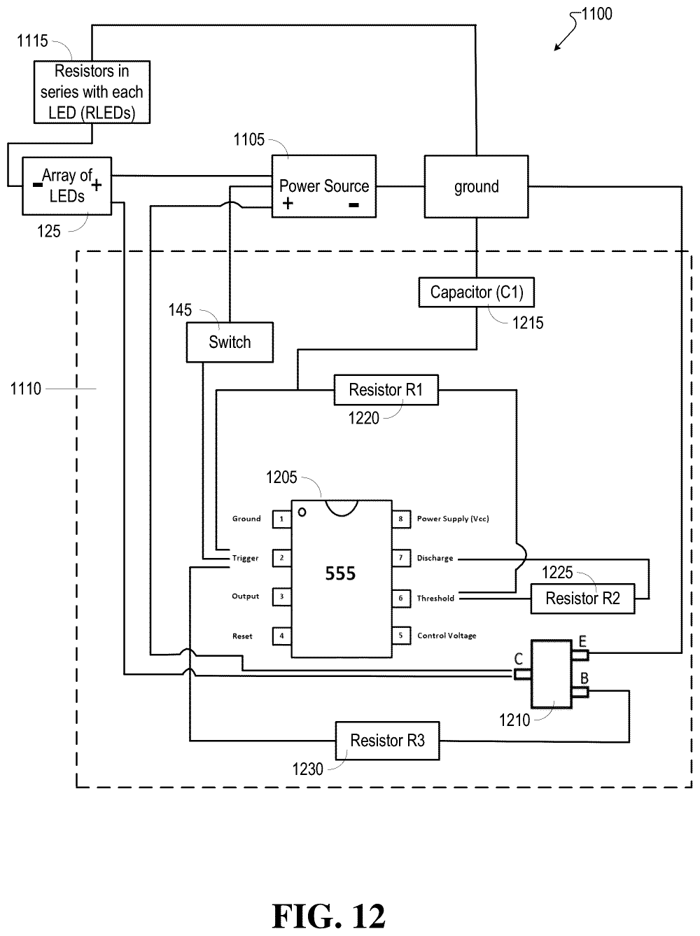

FIG. 12 illustrates an example of the circuit 1100 in which the timer IC 1110 uses a 555 timer IC 1205, which is available commercially, and an NPN transistor 1210, which is also available commercially, both of which are well known to those of ordinary skill in the art. The circuit 1100 will now be described operationally.

Initially, with the switch in the off position, the circuit is in a standby state, and the UV LEDs are off. When the user pushes the switch 145, it triggers the timer IC 1110. The trigger input (Pin 2) of the 555 timer IC 1205 receives a high signal, initiating the timing cycle. During the timing cycle, capacitor C1 1215 is charged as current flows through resistor R1 1220. As mentioned above with respect to FIG. 11, the current-limiting resistors (R_LED) in series with the UV LEDs help protect the LEDs from excess current. The NPN transistor 1210 can facilitate control of the activation and deactivation of the UV sources 125 based on the switch-triggered timing cycle. Resistor R2 1225 is primarily responsible for the timing function of the 555 timer IC 1205, while resistor R3 1230 controls the base current of the NPN transistor, enabling it to function as a switch that activates and deactivates the LED array based on the timing cycle initiated by the 555 timer IC 1205. When the timer cycle completes (i.e., when time is up), the UV LEDs turn off, and the circuit returns to a standby state until the user presses the switch 145 again to initiate another timing cycle.

Still referring to FIG. 12, by adjusting the values of the resistors and capacitors, the timing cycle can be modified to achieve durations of 1, 2, 5, 15, etc. minutes, while keeping the role of the NPN transistor 1210 as a switch to control the LED array based on the timing cycle. In example embodiments, the shoe sanitizer 100 can comprise a switch that is a knob in which the timing duration can be selectable from 1 minute, 5 minutes, 10 minutes, and 15 minutes. For these example embodiments, one can implement this with variable resistors (potentiometers) to allow users to adjust the timing cycle.

In particular and in regard to the various functions performed by the above described components, devices, circuits, systems and the like, any claim terms comprising a reference to a “means” used to describe such components are intended to correspond, unless otherwise indicated, to any component which performs the specified function of the described component (e.g., a functional equivalent), even though not structurally equivalent to the disclosed structure, which performs the function in the herein illustrated example aspects of the embodiments. Further, unless the term “means” appears in a claim, the claim is not to be defined or construed as a means plus function claim.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while a particular feature may have been disclosed with respect to only one of several implementations, such feature can be combined with one or more other features of the other implementations as may be desired and advantageous for any given or particular application. Furthermore, to the extent that the terms “have”, “having”, “includes” and “including” and variants thereof are used in either the detailed description or the claims, these terms are intended to be inclusive in a manner similar to the term “comprising.”

The above descriptions of various embodiments of the subject disclosure and corresponding figures and what is described in the Abstract, are described herein for illustrative purposes, and are not intended to be exhaustive or to limit the disclosed embodiments to the precise forms disclosed. It is to be understood that one of ordinary skill in the art can recognize that other embodiments comprising modifications, permutations, combinations, and additions can be implemented for performing the same, similar, alternative, or substitute functions of the disclosed subject matter, and are therefore considered within the scope of this disclosure.

For example, disclosed systems and apparatuses and components or subsets thereof (referred to hereinafter as components) should neither be presumed to be exclusive of other disclosed systems and apparatuses, nor should an apparatus be presumed to be exclusive to its depicted components in an example embodiment or embodiments of this disclosure, unless where clear from context to the contrary.

The scope of this disclosure is generally intended to encompass modifications of depicted embodiments with additions from other depicted embodiments, where suitable, interoperability among or between depicted embodiments, where suitable, as well as addition of a component(s) from one embodiment(s) within another or subtraction of a component(s) from any depicted embodiment, where suitable, aggregation of components (or embodiments) into a single component achieving aggregate functionality, where suitable, or distribution of functionality of a single system or component into multiple systems or components, where suitable. In addition, incorporation, combination or modification of systems or components depicted herein or modified as stated above with systems, apparatuses, components or subsets thereof not explicitly depicted herein but known in the art or made evident to one with ordinary skill in the art through the context disclosed herein are also considered within the scope of the present disclosure.

Therefore, the disclosed subject matter should not be limited to any single embodiment described herein, but rather should be construed in breadth and scope in accordance with the claims below.

What is claimed is:

1. A device, comprising:
a cover that can be placed over the mouth of a shoe, wherein:
the cover is attached to a central body, and wherein the central body comprises a plurality of ultraviolet light sources, the ultraviolet light sources operable to emit ultraviolet radiation;
the cover comprises a switch that controls power flow to the plurality of ultraviolet light sources;
the ultraviolet light sources are positioned so that the ultraviolet light emitted from the plurality of ultraviolet light sources can reach the front, back, and sides of the inside of the shoe; and
wherein the central body is extendable so that the central body can change in length to accommodate shoes of different dimensions.

2. The device of claim 1, wherein the power source can be located within the central body.

3. The device of claim 1, wherein the power source can be associated with the cover.

4. The device of claim 1, wherein the cover comprises a seal that enables the cover to retain its position covering the mouth of the shoe.

5. The device of claim 1, wherein the central body is connected to the cover via a connector.

6. The device of claim 1, wherein the central body has attached thereto a stabilizer that prevents the central body from coming into contact with an insole of the shoe.

7. The device of claim 1, wherein the stabilizer comprises a flexible material.

8. The device of claim 1, wherein the central body comprises openings through which UV light can pass through.

9. The device of claim 1, further comprising a timing circuit that controls the flow of power to the UV sources based on an elapsed amount of time.

10. The device of claim 9, wherein the elapsed amount of time depends upon the position of a knob.

11. A device, comprising:
a cover that can be placed over the mouth of a shoe, wherein:
the cover is attached to a central body, and wherein the central body comprises one or more ultraviolet light sources, the ultraviolet light source operable to emit ultraviolet radiation;
the cover comprises a switch that controls power flow to the one or more ultraviolet light sources;
the central body is connected to the cover via a connector;
the one or more ultraviolet light sources are positioned so that the ultraviolet light emitted from the one or more ultraviolet light sources can reach the front, back, and sides of the inside of the shoe; and
the central body is extendable so that the central body can change in length to accommodate shoes of different dimensions.

12. The device of claim 11, wherein the power source can be located within the central body.

13. The device of claim 11, wherein the power source can be located within the cover.

14. The device of claim 11, wherein the cover has attached to it a seal that facilitates the cover retaining its position covering the mouth of the shoe.

15. The device of claim 11, wherein the central body has attached thereto a stabilizer that prevents the central body from coming into contact with an insole of the shoe.

16. The device of claim 11, wherein the connector is extendable in length to accommodate shoes of varying height.

17. A device, comprising:
a cover that can be placed over the mouth of a shoe, wherein:
the cover is attached to a central body, and wherein the central body comprises one or more ultraviolet light sources, the ultraviolet light source operable to emit ultraviolet radiation;
the cover comprises a switch that controls power flow to the one or more ultraviolet light sources;
the central body is connected to the cover via a connector;
the central body has attached thereto a stabilizer that prevents the central body from coming into contact with an insole of the shoe.
the one or more ultraviolet light sources are positioned so that the ultraviolet light emitted from the one or more ultraviolet light sources can reach the front, back, and sides of the inside of the shoe; and
the central body is extendable so that the central body can change in length to accommodate shoes of different dimensions.

18. The device of claim 17, wherein the power source can be located within the central body.

19. The device of claim 17, wherein the power source can be located within the cover.

20. The device of claim 17, wherein the cover has attached to it a seal that allows the cover to retain its position covering the mouth of the shoe.
